(19) United States
(12) Patent Application Publication
(10) Pub. No.: US 2016/0183992 A1
(43) Pub. Date: Jun. 30, 2016

(54) INTERNAL FIXATION DEVICE FOR BONE FRACTURES

(71) Applicant: METAL INDUSTRIES RESEARCH AND DEVELOPMENT CENTRE, Kaohsiung (TW)

(72) Inventors: Shih-Hua Huang, Kaohsiung (TW); Tung-Lin Tsai, Kaohsiung (TW); Chung-Kuei Hsueh, Kaohsiung (TW); Pei-Yuan Lee, Kaohsiung (TW)

(21) Appl. No.: 14/584,723

(22) Filed: Dec. 29, 2014

(51) Int. Cl. A61B 17/86

(52) U.S. Cl. CPC A61B 17/863

(57) ABSTRACT

An internal fixation device is adapted for fixing a first bone fracture to a second bone fracture. The internal fixation device includes: a shank adapted to extend into the first and second bone fractures and including a connecting segment that is made of a shape-memory alloy, and a first threaded segment that is connected to a first end of the connecting segment and that is adapted to be fixed in the first bone fracture; and a fixing unit adapted to fasten the second bone fracture to the first bone fracture. When environmental temperature is increased to a predetermined temperature, the connecting segment is shortened such that the fracture surfaces of the first and second bone fractures are 15 pressed closely against each other.

### FIELD OF THE INVENTION

This invention relates to an internal fixation device adapted for fixing a first bone fracture to a second bone fracture.

### BACKGROUND OF THE INVENTION

A femoral neck fracture or a scaphoid fracture is usually caused by a torsional force, thereby forming a spiral fracture or an oblique fracture. Internal fixation is one of the methods of treatment for the aforesaid fractures.

Referring to FIG. 1, two conventional internal fixation devices 1 are adapted for fixing a first bone fracture 2 to a second bone fracture 2′. The first bone fracture 2 has a fracture surface 21. The second bone fracture 2′ has a fracture surface 21′ complementary to the fracture surface 21 of the first bone fracture 2.

Each of the internal fixation devices 1 includes a head 12, a threaded segment 13, and a connecting segment 11 that interconnects the head 12 and the threaded segment 13 and that is made of metal. Each internal fixation device 1 is used to permit the fracture surfaces 21, 21′ of the first and second bone fractures 2, 2′ to press against each other through a compression force generated by cooperation of the head 12 and the threaded segment 13 as the head 12 abuts against an outer surface 22′ of the second bone fracture 2′ and the threaded segment 13 is fixed in the first bone fracture 2.

However, factors such as decrease in bone mass and density, failure in surgery, or shifting of the internal fixation devices 1 may result in formation of gaps (not 5 shown) between the internal fixation devices 1 and the first and second bone fractures 2, 2′. As such, cooperation of the head 12 and the threaded segment 13 can no longer permit the fracture surfaces 21, 21′ to press closely against each other, and a clearance (not shown) may be formed between the fracture surfaces 21, 21′ of the first and second bone fractures 2, 2′ , thereby resulting in formation of scar tissue therein. In addition, when the clearance becomes wider, a patient may experience persistent discomfort and bone healing may be adversely affected.

Since the elastic modulus of the internal fixation devices 1 is larger than that of the first and second bone fractures 2, 2′ , the internal fixation devices 1 can bear more stresses from the outside and therefore the first and second bone fractures 2, 2′ are shielded from stress, which is known as a stress-shielding effect. Because of the stress-shielding effect, the first and second bone fractures 2, 2′ may not receive enough stresses for the growth of new bone tissue during healing, and may result in atrophy of the first and second bone fractures 2, 2′. As such, the internal fixation device may be loosened.

### SUMMARY OF THE INVENTION

Therefore, an object of the present invention is to provide an internal fixation device that allows a fracture surface of a first bone fracture and a fracture surface of a second bone fracture to be constantly and closely pressed against each other.

According to the present invention, an internal fixation device is adapted for fixing a first bone fracture to a second bone fracture, the first bone fracture having a fracture surface, the second bone fracture having a fracture surface complementary to the fracture surface of the first bone fracture. The internal fixation device includes a shank adapted to extend into the first and second bone fractures, and a fixing unit adapted to fasten the second bone fracture to the first bone fracture. The shank includes a connecting segment that is made of a shape-memory alloy and that has a first end, and a first threaded segment that is connected to the first end of the connecting segment and that is adapted to be fixed in the first bone fracture; wherein, when environmental temperature is increased to a predetermined temperature, the connecting segment is shortened such that the fracture surfaces of the first and second bone fractures are pressed closely against each other.

### BRIEF DESCRIPTION OF THE DRAWINGS

Other features and advantages of the present invention will become apparent in the following detailed description of the embodiments of this invention, with reference to the accompanying drawings, of which:

FIG. 1 is a schematic view showing two conventional internal fixation devices which fasten a first bone fracture and a second bone fracture;

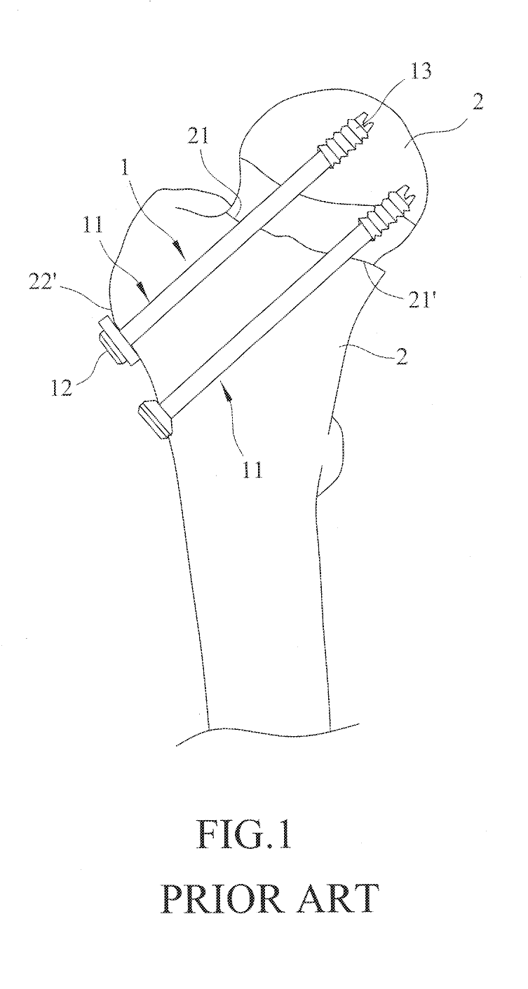

FIG. 2 is a perspective view of the first embodiment of an internal fixation device according to the present invention;

FIG. 3 is a schematic view showing that the internal fixation device of the first Embodiment fastens a first bone fracture to a second bone fracture; and

FIG. 4 is a schematic view of two internal fixation devices of the second embodiments according to the present invention, showing that the first bone fracture and the second bone fracture are fastened to each other.

### DETAILED DESCRIPTION OF THE EMBODIMENTS

Before the present invention is described in greater detail, it should be noted that like elements are denoted by the same reference numerals throughout the disclosure.

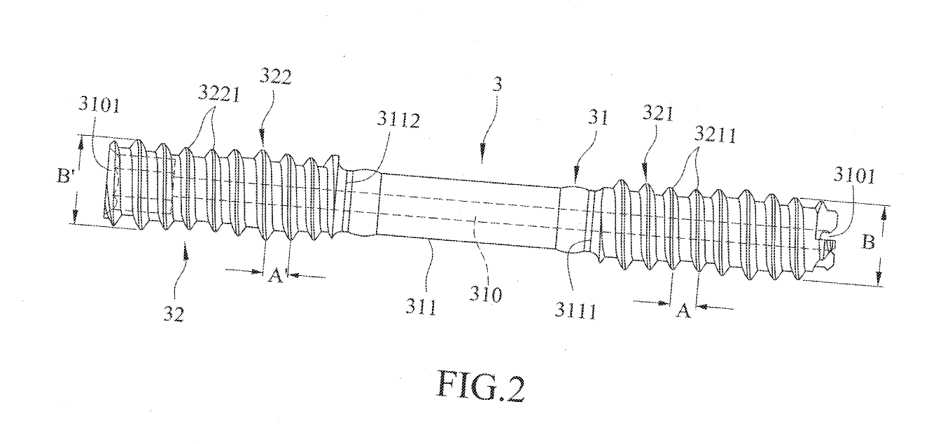

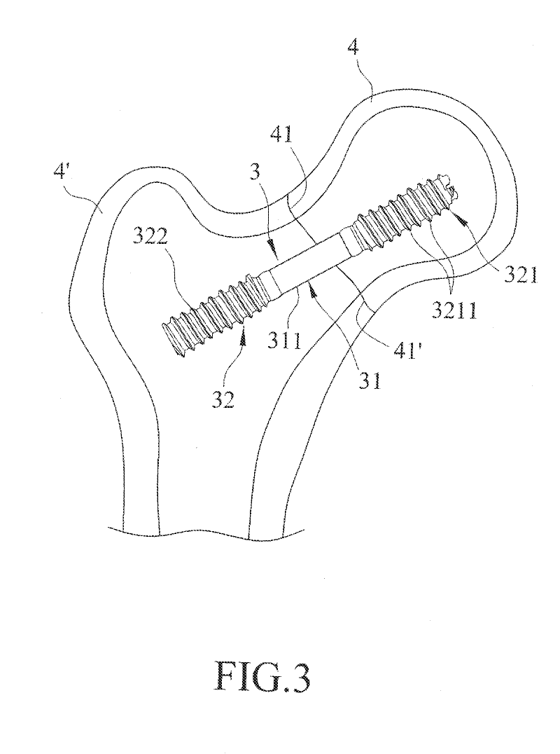

Referring to FIGS. 2 and 3, the first embodiment of an internal fixation device 3 According to the present invention is adapted for fixing a first bone fracture 4 to a second bone fracture 4′. The first bone fracture 4 has a fracture surface 41. The second bone fracture 4′ has a fracture surface 41′ complementary to the fracture surface 41 of the first bone fracture 4.

The internal fixation device 3 includes a shank 31 adapted to extend into the first and second bone fractures 4, 4′, and a fixing unit 32 adapted to fasten the second bone fracture 4′ to the first bone fracture 4.

The shank 31 includes a connecting segment 311, a first threaded segment 321 and an internal passage 310. The connecting segment 311 is made of a shape-memory alloy and has opposite first and second ends 3111, 3112. The first threaded segment 321 is integrally connected to the first end 3111 and is adapted to be fixed in the first bone fracture 4. The internal passage 310 has two opposite open ends 3101 and extends along the length of the shank 31. The fixing unit 32 is configured as a second threaded segment 322 that is integrally connected to the second end 3112 of the connecting segment 311 and that is adapted to be fixed in the second bone fracture 4′. In practice, 15 the internal fixation device 3 is made of the shape-memory alloy.

The connecting segment 311 further has a smooth outer surface that is adapted to extend through the fracture surfaces 41, 41′ of the first and second bone fractures 4, 4′ and that allows the first and second bone fractures 4, 4′ to slide easily thereon, thereby allow the fracture surfaces 41, 41′ to press easily and closely against each other for internal fixation of the first and second bone fractures 4, 4′.

In this embodiment, the first threaded segment 321 has a plurality of thread turns 3211, and at least two of pitches (A) defined by the thread turns 3211 are different from each other. The second threaded segment 322 has a plurality of thread turns 3221, and at least two of pitches (A′) defined by the thread turns 3221 are different from each other. The thread turns 3211, 3221 with variable pitches (A) , (A′) allow the first threaded segment 321 and the second threaded segment 322 to be more firmly fixed in the first bone fracture 4 and the second bone fracture 4′, respectively, thereby preventing the internal fixation device 3 from loosening.

In this embodiment, at least two of outer diameters (B) of the thread turns 3211 of the first threaded segment 321 are different from each other, and at least two of outer diameters (B′) of the thread turns 3221 of the second threaded segment 322 are different from each other. The 15 thread turns 3211, 3221 with variable outer diameters (B), (B′) permit a more even distribution of external stresses on the first and second bone fractures 4, 4′, thereby avoiding atrophy of the first and second bone fractures 4, 4′caused by stress shielding.

Shape-memory alloy can be deformed when cooled to a temperature below its phase transition temperature, and is capable of returning to its pre-deformed shape when heated to a temperature above the phase transition temperature. Since the connecting segment 311 is made of the shape-memory alloy, when environmental temperature is increased to a predetermined temperature which is higher than the phase transition temperature of the shape-memory alloy, the connecting segment 311 is shortened to create a pulling force along the length of the shank 31 such that the fracture surfaces 41, 41′ of the first and second bone fractures 4, 4′ are pressed closely against each other, thereby facilitating bone healing. To be specific, in this embodiment, the phase transition temperature of the shape-memory alloy is set to be the temperature of the human body.

The shape-memory alloy is, for example but not limited to, a nickel-titanium based alloy, a copper-based alloy or an iron-based alloy. Because the nickel-titanium based alloy has excellent stability, fatigue resistance and biocompatibility, it is used in the first embodiment. Additionally, removal of the internal fixation device 3 after operation is not necessary due to the biocompatibility of the nickel-titanium based alloy, and the presence of the internal fixation device 3 in a patient's body does not affect medical examination such as MRI or CT. A method of producing the nickel-titanium based alloy includes the steps of (a) melting nickel and titanium with a weight ratio of about 1:1 by vacuum arc melting, and (b) applying a heat treatment to adjust mechanical properties and for shape memory treatment.

During an operation of internal fixation, the internal passage 310 is adapted for insertion of a guide pin (not shown) which guides movement of the first embodiment precisely toward a predetermined position. Therefore, the internal passage 310 can enhance precision in positioning of the first embodiment so as to facilitate bone healing. Additionally, the design of the internal passage 310 permits the first embodiment to be applied in minimally invasive surgery.

Moreover, the first embodiment can be completely disposed within the first and second bone fractures 4, 4′ without protruding out of the first and second bone fractures 4, 4′, thereby avoiding stimulation of soft tissues and articular facets. As such, the first embodiment is suitable for fixation of intra-articular fracture.

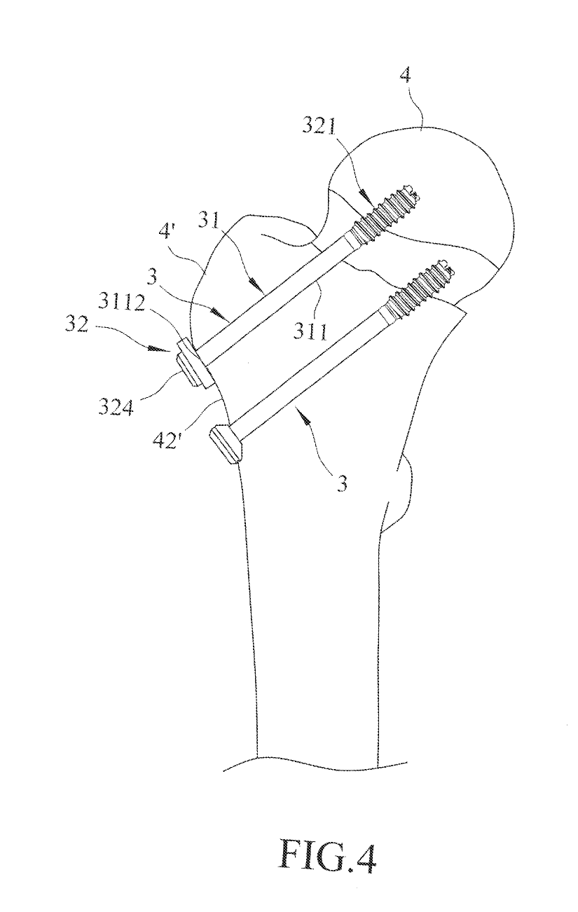

Referring to FIG. 4, the second embodiment of an internal fixation device according to the present invention is similar to that of the first embodiment. The difference between the first and second embodiments resides in that the fixing unit 32 is configured as a head 324 that is integrally connected to the second end 3112 of the connecting segment 311 and that is adapted to abut against an outer surface 42′ of the second bone fracture 4′ when the first threaded segment 321 is fixed in the first bone fracture 4. As shown in FIG. 4, the first bone fracture 4 is fixed to the second bone fracture 4′ using two internal fixation devices 3. In the second embodiment, the head 324 is a screw head. Comparing to the first embodiment, the second embodiment is easier to produce and provides an alternative choice in practice.

To sum up, since the connecting segment 311 is made of the shape-memory alloy, the connecting segment 311 is shortened such that the fracture surfaces 41, 41′ of the first and second bone fractures 4, 4′ are pressed closely against each other when environmental temperature is increased to the predetermined temperature, thereby enhancing fixation of the first bone fracture 4 to the second bone fracture 4′, and facilitating bone healing. In addition, the thread turns 3211, 3221 with variable 10 pitches (A) , (A′) allow the first and second threaded segments 321, 322 to be more firmly fixed in the first and second bone fractures 4, 4′, respectively. Moreover, the thread turns 3211, 3221 with variable outer diameters (B), (B′) permit external stresses to be distributed more evenly to avoid atrophy of the first and second bone fractures 4, 4′.

While the present invention has been described in connection with what is considered the most practical embodiments, it is understood that this invention is not limited to the disclosed embodiments but are intended to cover various arrangements included within the spirit and scope of the broadest interpretation and equivalent arrangements.

What is claimed is:

1. An internal fixation device adapted for fixing a first bone fracture to a second bone fracture, the first bone fracture having a fracture surface, the second bone fracture having a fracture surface complementary to the fracture surface of the first bone fracture, said internal fixation device comprising:
a shank adapted to extend into the first and second bone fractures and including a connecting segment that is made of a shape-memory alloy and that has a first end, and a first threaded segment that is connected to said first end of said connecting segment and that is adapted to be fixed in the first bone fracture; and
a fixing unit adapted to fasten the second bone fracture to the first bone fracture;
wherein, when environmental temperature is increased to a predetermined temperature, said connecting segment is shortened such that the fracture surfaces of the first and second bone fractures are pressed closely against each other.

2. The internal fixation device as claimed in claim 1, wherein said connecting segment further has a smooth outer surface adapted to extend through the fracture surfaces of the first and second bone fractures.

3. The internal fixation device as claimed in claim 2, wherein said connecting segment further has a second end that is opposite to said first end, said fixing unit being configured as a head that is integrally connected to said second end of said connecting segment and that is adapted to abut against an outer surface of the second bone fracture when said first threaded segment is fixed in the first bone fracture.

4. The internal fixation device as claimed in claim 3, wherein said first threaded segment has a plurality of thread turns, at least two of pitches defined by said thread turns being different from each other.

5. The internal fixation device as claimed in claim 4, wherein at least two of outer diameters of said thread turns of said first threaded segment are different from each other.

6. The internal fixation device as claimed in claim 5, wherein said shape-memory alloy is a nickel-titanium based alloy.

7. The internal fixation device as claimed in claim 2, wherein said connecting segment further has a second end that is opposite to said first end, said fixing unit being configured as a second threaded segment that is integrally connected to said second end of said connecting segment.

8. The internal fixation device as claimed in claim 7, wherein said second threaded segment has a plurality of thread turns, at least two of pitches defined by said thread turns being different from each other.

9. The internal fixation device as claimed in claim 8, wherein at least two of outer diameters of said thread turns of said second threaded segment are different from each other.

10. The internal fixation device as claimed in claim 9, wherein said shank further includes an internal passage that has two opposite open ends and that extends along length of said shank.

11. The internal fixation device as claimed in claim 10, wherein said shape-memory alloy is a nickel-titanium based alloy.

12. The internal fixation device as claimed in claim 1, wherein said connecting segment further has a second end that is opposite to said first end, said fixing unit being configured as a head that is integrally connected to said second end of said connecting segment and that is adapted to abut against an outer surface of the second bone fracture when said first threaded segment is fixed in the first bone fracture.

13. The internal fixation device as claimed in claim 1, wherein said shank further includes an internal passage that has two opposite open ends and that extends along length of said shank.

14. The internal fixation device as claimed in claim 1, 10 wherein said first threaded segment has a plurality of thread turns, at least two of pitches defined by said thread turns being different from each other.

15. The internal fixation device as claimed in claim 1, wherein said first threaded segment has a plurality of thread turns, at least two of outer diameters of said thread turns of said first threaded segment being different from each other.

16. The internal fixation device as claimed in claim 1, wherein said shape-memory alloy is a nickel-titanium based alloy.

17. The internal fixation device as claimed in claim 1, wherein said connecting segment further has a second end that is opposite to said first end, said fixing unit being configured as a second threaded segment that is integrally connected to said second end of said connecting segment.

18. The bone-fastening implant as claimed in claim 17, wherein said second threaded segment has a plurality of thread turns, at least two of pitches defined by said thread turns being different from each other.

19. The internal fixation device as claimed in claim 18, wherein at least two of outer diameters of said thread turns of said second threaded segment are different from each other.
